(19) United States
(12) Patent Application Publication
(10) Pub. No.: US 2005/0020980 A1
(43) Pub. Date: Jan. 27, 2005

(54) Coupling system for an infusion pump

(76) Inventors: Yoshio Inoue, Osaka (JP); Kirk Ramey, Bedford, VA (US); Yoshiyuki Sonoda, Osaka (JP); Robert Sowell, Boca Raton, FL (US); Robert Williams, Niceville, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 10/863,895

(22) Filed: Jun. 7, 2004

Related U.S. Application Data
(60) Provisional application No. 60/476,973, filed on Jun. 9, 2003.

(51) Int. Cl. A61M 1/00

(52) U.S. Cl. 604/152

(57) ABSTRACT

A pump system for an infusion system includes a linear drive (36, 36′) which minimizes the space occupied by the pump components in a portable housing (10, 10′). A motor (34) and a motor drive shaft (42) are arranged in parallel with, and adjacent to a syringe (14, 14′) and lead screw (94, 94′). A gear box (54) connects the drive shaft and lead screw to transfer rotational movements between them. A piston driving member, such as a drive nut (116) converts the rotational movement of the lead screw into linear motion of a syringe piston (24). A cap (190, 190′) couples the syringe (14, 14′) to the housing and provides an outlet for the liquid to be dispensed. In one embodiment, the cap (190′) is configured to rotate relative to the housing in one direction only, during locking. Rotational movement is also used for locking the piston (24) to the drive nut (116) against relative axial movement. In another embodiment, the cap (190′) carries a rotatable hub (330) which is connected at a first end (336) with an infusion line (191) and at a second end defines a needle (338) for piercing a closure (340) on the syringe.

### CROSS-REFERENCE TO RELATED APPLICATION

This application is related to and claims priority to U.S. Provisional Patent Application Ser. No. 60/476,973, filed Jun. 09, 2003, entitled COUPLING SYSTEM FOR AN INFUSION PUMP, which application claims the priority of U.S. application Ser. No. 10/121,318, filed Apr. 12, 2002, entitled DRIVE SYSTEM FOR AN INFUSION PUMP, which is incorporated herein in its entirety by reference, and U.S. Provisional Application Ser. No. 60/283,815, filed Apr. 13, 2001, also incorporated herein in its entirety by reference, the entirety of all of which is incorporated herein by reference.

### STATEMENT REGARDING FEDERALLY SPONSORED RESEARCH OR DEVELOPMENT

n/a

### FIELD OF THE INVENTION

The present invention relates to a method and system for delivering medicament, such as insulin, from a syringe, and more particularly, to a portable pump having a coupling system for allowing the syringe to be locked to a syringe housing and a piston of the syringe to be locked to a drive nut in the same rotational movement. It should be appreciated, however, that the invention also has application in the miniaturization of pumps for delivery of other liquid substances.

### BACKGROUND OF THE INVENTION

Pump systems which use a piston-operated cartridge for delivery of a medicament, such as insulin, allow patients to administer safely doses of an intravenous or subcutaneous medication at will, without the need for constant supervision by medical staff. These devices often include a housing, which is small enough to fit in a patient's pocket, that houses the cartridge, a motor, and a drive system. A compact power supply, such as a rechargeable battery, is also included for supplying power to the motor. The outside of the housing provides key pad entry for allowing the patient to enter data such as to program the rate of insulin delivery and to modify the delivery rate according to the patient's expected or actual carbohydrate intake.

The cartridge of insulin is replaced or refilled at intervals. In conventional systems, this is often a complex operation, requiring considerable dexterity on the part of the user. If the cartridge insertion operation is not performed correctly, the cartridge may be improperly positioned with respect to the drive system, and inaccurate dosages administered as a result.

The present invention provides for a new and improved pump system, which overcomes the above-referenced problems, and others.

### SUMMARY OF THE INVENTION

In accordance with one aspect of the present invention, a liquid delivery system is provided. The system includes a housing which accommodates a syringe containing the liquid. Means are provided for expelling a liquid from the syringe carried by the housing. A cap selectively connects the syringe with the housing and provides a fluid passage between the syringe and a fluid line when the fluid line is connected with the cap. The cap includes means for selectively connecting the cap with the syringe. There are at least two spaced projections on one of the cap and the housing. There are at least two spaced slots on the other of the cap and the housing which receive the projections. When the projections are positioned in the slots, the cap is moved relative to the housing in a locking direction to lock the cap to the housing.

In accordance with another aspect of the present invention, a cap for connecting a syringe to a housing of an infusion system is provided. The cap includes a luer connection for selective interconnection with an outlet port of the syringe. The luer connection includes an interior passage which fluidly connects the outlet port with an infusion line when the infusion line is connected with the cap. A skirt is radially outwardly spaced from the luer connection. The skirt includes first and second arcuately spaced projections for engagement with first and second arcuately spaced slots on the housing. When the projections are positioned in the slots, the cap is rotatable relative to the housing in a locking direction to lock the cap to the housing.

In accordance with another aspect of the present invention, a method of assembling an infusion system is provided. The method includes coupling a cassette, containing a liquid to be infused, to a cap. The cap is mounted on a housing such that the cassette is received within the housing. The mounting step includes engaging first and second projections on one of the cap and the housing with first and second slots on the other of the cap and the housing, the projections being configured such that the first projection is capable of being received only in the first slot. The cap is rotated, relative to the housing in a locking direction to lock the cap to the housing.

One advantage of at least one embodiment of the present invention is that a syringe is coupled to a pump housing in the same movement as a drive nut of the drive system is coupled to a piston of the syringe.

Another advantage of at least one embodiment of the present invention is that it reduces the size of an infusion pump for improved portability.

Another advantage of at least one embodiment of the present invention is that occlusions in an infusion line are detected.

Yet another advantage of at least one embodiment of the present invention is that the ravel of the drive mechanism is detected.

Still further advantages of the present invention will become apparent to those of skill in the art upon reading and understanding the following detailed description of the preferred embodiments.

### DESCRIPTION OF THE DRAWINGS

A more complete understanding of the present invention, and the attendant advantages and features thereof, will be more readily understood by reference to the following detailed ion when considered in conjunction with the accompanying drawings wherein:

The invention may take form in various components and arrangements of components, various steps and arrangements of steps. The drawings are only for purposes of illustrating preferred embodiments and are not to be construed as limiting the invention.

FIG. 1 is a side sectional view of an infusion pump system according to the invention, with the gear box removed;

FIG. 2 is a side sectional view of the lead screw, gear box, drive shaft, and motor fusion pump system of FIG. 1;

FIG. 3 is a schematic view of the gear box of FIG. 1;

FIG. 4 is an enlarged view of the yoke and lead screw of FIG. 1;

FIG. 5 is an enlarged sectional view of the lead screw and piston of FIG. 1, in engaged position;

FIG. 6 is an enlarged side sectional view of the piston drive member of FIG. 1;

FIG. 7 is an enlarged front perspective view of the piston drive member of FIG. 6, showing the position of a sensor;

FIG. 8 is an enlarged rear perspective view of the piston drive member of FIG. 6;

FIG. 9 is a side perspective view of the piston of FIG. 1;

FIG. 10 is an elevational view of the piston viewed generally from the right-hand end of FIG. 9;

FIG. 11 is an enlarged side view of the barrel of FIG. 1;

FIG. 12 is a side sectional view of the barrel of FIG. 11;

FIG. 13 is an enlarged side sectional view of the barrel of FIG. 11;

FIG. 14 is an enlarged elevational view of the cap of FIG. 1;

FIG. 15 is a side view of the cap of FIG. 1;

FIG. 16 is a top plan view of the cap of FIG. 1;

FIG. 17 is a side sectional view through B-B of the cap of FIG. 14;

FIG. 18 is a side sectional view through A-A of the cap of FIG. 14;

FIG. 19 is an enlarged side sectional view of the cap of FIG. 18;

FIG. 20 is a side view of the housing and cap of FIG. 1;

FIG. 21 is a side view of the housing and cap of FIG. 20;

FIG. 22 is an enlarged perspective view of the housing of FIG. 1, showing the ion for the syringe cap;

FIG. 23 is a further enlarged perspective view of the housing of FIG. 1, showing the connection for the syringe cap;

FIG. 24 is a side sectional view of a second embodiment of an infusion pump drive system and syringe; and

FIG. 25 is a third embodiment of an infusion pump drive system and syringe.

FIG. 26 is a cross sectional view of another embodiment of an infusion pump system according to the present invention;

FIG. 27 is a perspective view of the infusion pump system of FIG. 26;

FIG. 28 is an enlarged perspective view of the cap of FIG. 26;

FIG. 29 is another perspective view of the cap of FIG. 28, showing a needle;

FIG. 30 is a side sectional view of the cap of FIG. 28, attached to a syringe;

FIG. 31 is a perspective view of the cap and syringe of FIG. 30, showing a connector for connecting the piston to a drivenut;

FIG. 32 is a side view of the cap and syringe of FIG. 31;

FIG. 33 is a side view of the cap and syringe of FIG. 32, showing the hub rotated through 90 degrees.

FIG. 34 is a side sectional view of the cap and syringe of FIG. 32;

FIG. 35 is an enlarged perspective view of the housing, cap, and syringe of FIG. 26, during insertion of the syringe; and

FIG. 36 is another perspective view of the housing, cap, and syringe of FIG. 26, during insertion of the syringe.

### DETAILED DESCRIPTION OF THE INVENTION

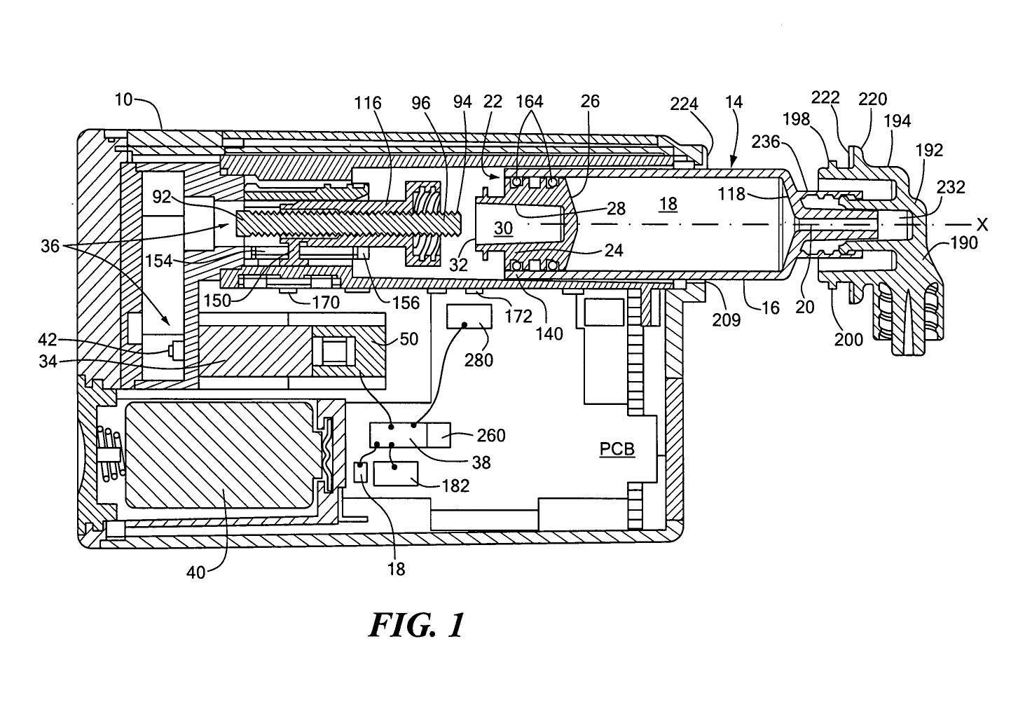

With reference to FIG. 1, a portable pump system for use in an ambulatory injection system, such as an insulin injection system, is shown. The system includes a housing 10, which is designed to fit conveniently in the pocket of a user or to be attached to a belt clip. A cassette 14, such as a disposable or reusable syringe, is selectively received within the housing 10. FIG. 1 shows the syringe 14 partially inserted into the housing 10. The syringe 14 holds a supply of a medicament, such as insulin, for injection into a diabetic patient, or other user in need of the medicament. The syringe 14 includes a barrel 16, which defines an internal chamber 18 for holding the medicament, a dispensing outlet 20 in fluid communication with the internal chamber and connected with one end of the barrel 16, and an opening 22 at an opposite end of the barrel 16. A plunger or piston 24 is received within the barrel 16 via the opening 22 for reciprocal motion within the barrel 16 for selectively ejecting the medicament from the barrel. The piston 24 includes a head portion or cap 26, which seals the opening 22, and a connection portion 28, extending from the head portion. An internal piston chamber 30 is formed in the piston, with an open end 32 furthest from the barrel 16.

Mounted within the housing 10, are a motor 34 and a drive system 36 for incrementally advancing the piston 24 to eject aliquots of the medicament, for example, according to a preprogrammed injection schedule. The motor 34 is under the control of a microprocessor-controller 38, which is preferably housed within the housing 10. Power for the motor and other operative components of the pump system is supplied by a replaceable/rechargeable battery 40, or other source of power. The motor 34 is preferably a stepper motor, which rotates in finite, small increments or steps. The drive system 36 includes a drive shaft 42, which is coupled to the motor so that it rotates a small portion of a revolution with each step of the motor. For example, the motor 34 may advance twenty steps to turn the drive shaft 42 one complete revolution, although other ratios may be contemplated or used without departing from the scope and intent of the present invention. As shown in FIG. 1, the drive shaft 42 is aligned generally in parallel with the longitudinal axis x of the syringe barrel 16 and piston 24 and rotates about an axis parallel with the x axis. It is also contemplated that the drive shaft may be coaxial with the piston axis of travel. However, an offset arrangement is desirable because of the ability to design a compact drive system.

In one embodiment, the power supply used to power the stepper motor 34 is a programmable power supply. Advantageously, the power supply in this embodiment can be programmed to vary the torque output of stepper motor 34. For example, an increase in the output voltage of the power supply increases the torque of motor 34 while a decrease in the output voltage lowers the torque of the motor. Control of the amount of motor torque is important for several reasons. First, there must be a sufficient amount of torque to ensure that the thrust of piston 24 is large enough to deliver the medicament to the user in a normal fashion but not too large so as to force medicament to leak out of the housing. Second, the torque of the motor must be sufficiently high so that the motor does not stall when operating at high speeds such as, for example, when the drive nut 116 is retracted. Finally, if an occlusion occurs, a high torque, i.e. a torque higher than that used to deliver medicament during normal operating conditions, is necessary to clear the occlusion. The programmable power supply therefore allows the user to alter the torque of motor 34 to account for any of the above occurrences.

An encoder 50 is operatively associated with an armature of the motor 34 to detect when the steps are occurring. A one or a multi-phase encoder may be used. A single-phase encoder detects the rotation of the motor. A two or multi-phase encoder alternatively registers a “zero” or a “one” output with each successive step and is capable of detecting not only the rotation of the motor but also the direction the motor is rotating in, i.e. clockwise or counterclockwise. The microprocessor-controller 38 is equipped with processing software or hardware to detect the change in output of the encoder and thereby determine whether the motor 34 is advancing as instructed. The microprocessor-controller 38 uses a measure of the number of motor steps to determine the rate and/or amount of medicament delivered. For example, it may instruct the motor to advance a selected number of steps over a certain time period, which equates to a determined volume of insulin ejected from the syringe in the selected time.

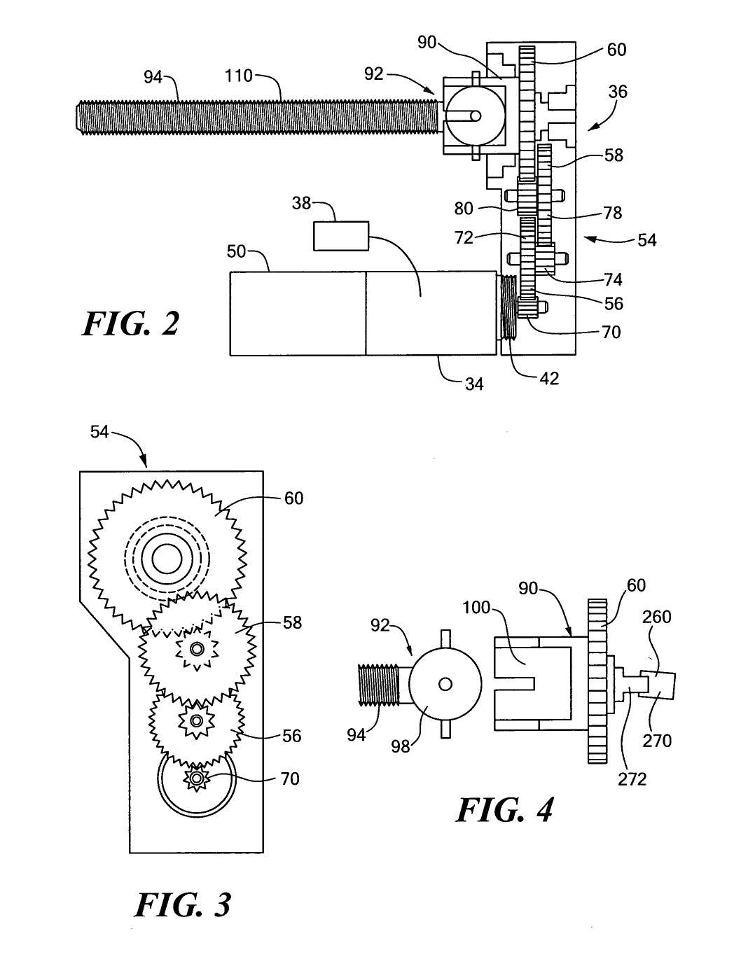

The drive shaft 42 drives a gearbox 54 comprising a series of gears 56, 58, 60, as shown in greater detail in FIG. 2 to transfer driving movement from the motor to the piston. The number and size of the gears will depend on the desired ratio of drive shaft rotation to output rotation.

As shown in FIGS. 2 and 3, the gearbox 54, by way of example has three gears 56, 58, and 60. Gears 56 and 58 are cluster gears, which each have a larger spur portion and a smaller pinion portion connected thereto. As shown in FIG. 2, the drive shaft 42 has a toothed portion 70 at its distal end, which drives a spur 72 of the gear 56, thereby turning an associated toothed pinion 74. The pinion 74 in turn engages a toothed spur 78 of the second gear 58, which in turn drivingly engages a toothed pinion 80 of the second gear. The pinion 80 engages teeth on the third gear 60, which forms a part of a universal yoke element 90. Again, the gear assembly is preferred because of the flexibility in designing a compact, reliable drive system.

As shown in FIG. 4, the yoke element 90 is connected with a first portion, or driven end 92 of a threaded, rotatable shaft or lead screw 94. Thus, the rotations of the motor shaft 42 are transferred to the lead screw via the gear box 54 at a selected ratio, for example a ratio of from about 30:1-100:1 (30 to 100 rotations of the motor shaft for each rotation of the lead screw) although varying ratios are also contemplated. A second, or distal end 96 (FIG. 1) of the lead screw 94 indirectly drives the piston 24 towards the chamber, so that the medicament is expelled.

The lead screw 94 is received longitudinally within the piston chamber 30 and extends generally parallel to the drive shaft 42. As shown in FIG. 4, the driven end 92 may comprise a ball and pin member 98, which is received in a slotted opening 100 in the yoke element 90. Other engagement methods, which transfer the rotation of the yoke member to the lead screw, are also contemplated, such as a fitting comprising a hexagonal pin (not shown) on the driven end 92, which is received in a corresponding hexagonal socket (not shown) in the universal joint 90 (not shown). Alternatively, the yoke 90 and lead screw 94 may be formed as a single component. The lead screw can be fixed to the gear box or disconnectable.

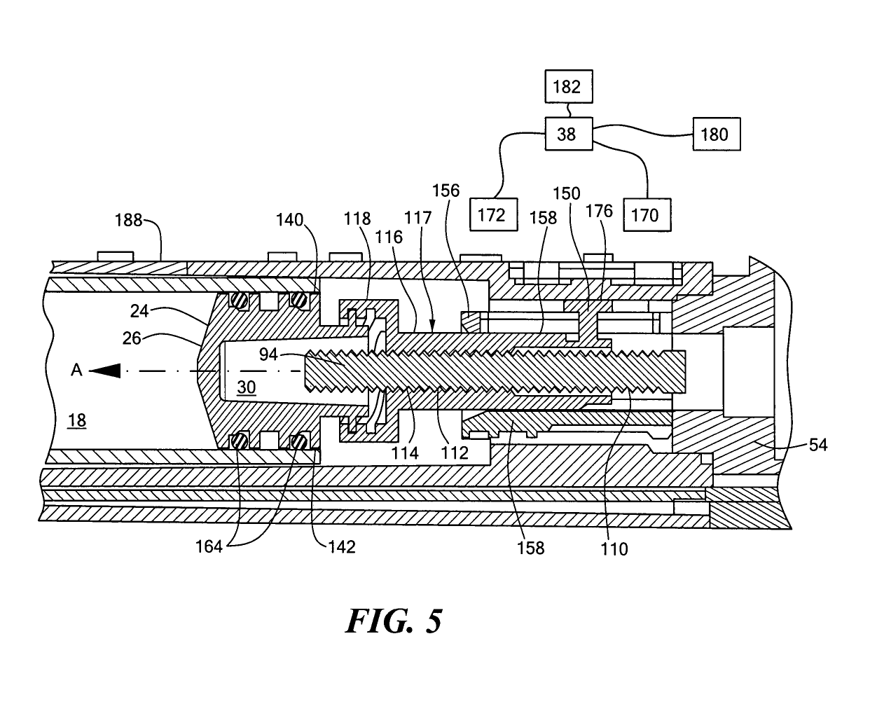

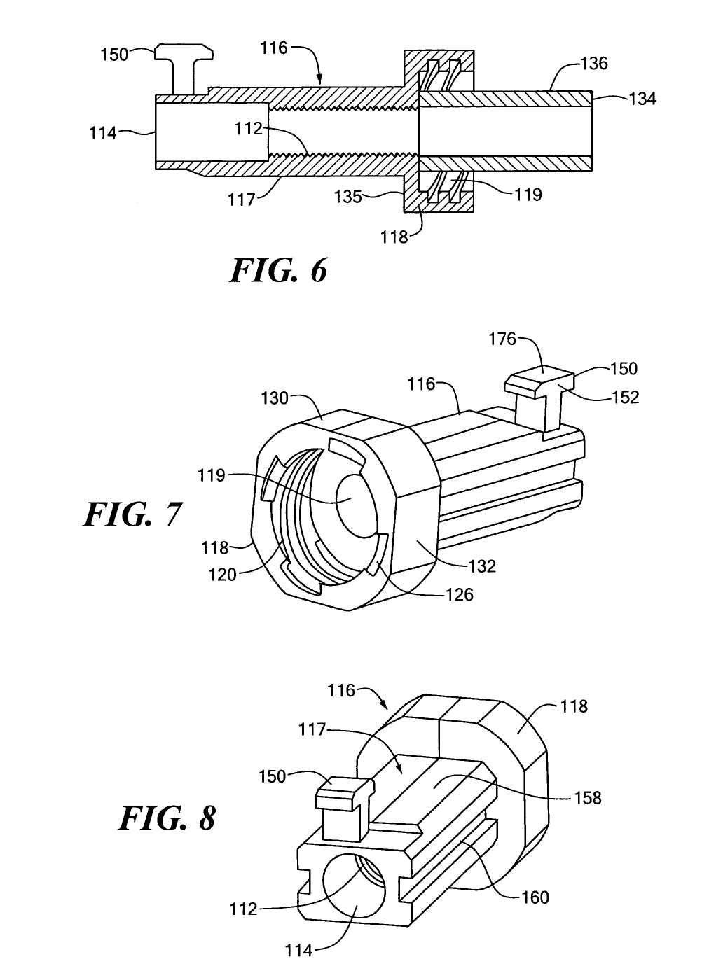

With reference now to FIGS. 5-8, the lead screw 94 is exteriorly threaded along at least a portion of its length. The external threads 110 engage corresponding threads 112 on an interior axial bore 114 of a drive nut or piston drive member 116, best shown in FIGS. 6-8. The pitch on the threads 110, 112 is such that as the lead screw rotates, the drive nut 116 moves towards the barrel chamber 18, in the direction of arrow A (FIG. 5) carrying the piston 24 with it. In particular, as the lead screw 94 is rotated in a driving direction, the drive nut 116 converts the rotational movement of the lead screw into a linear advancement of the drive nut 116 and piston 24 in a fluid expelling direction.

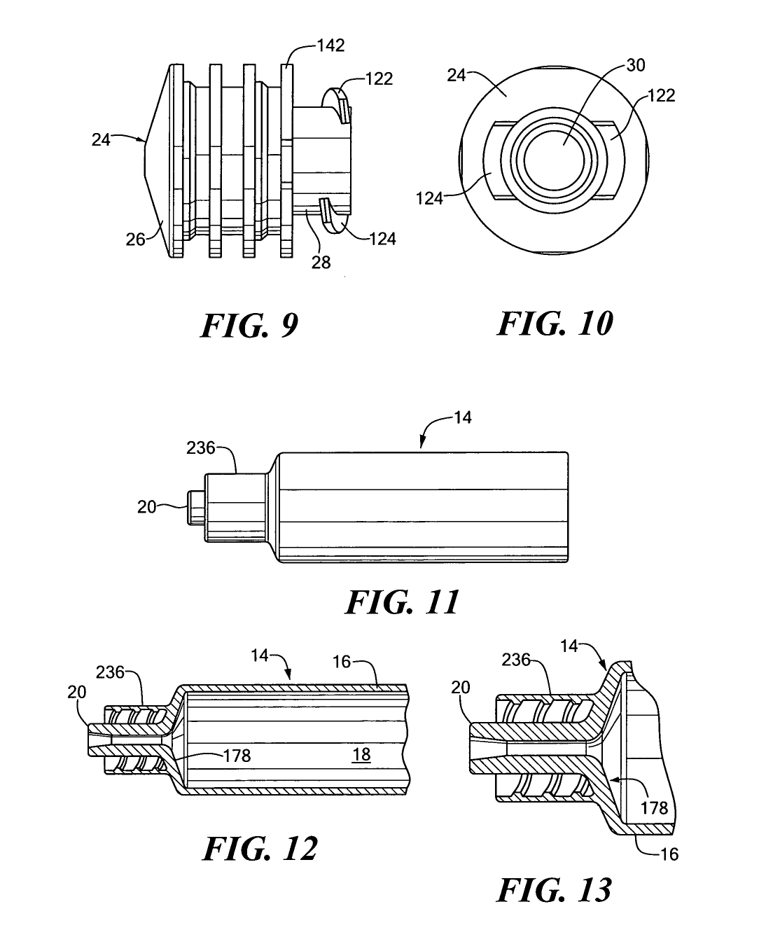

With continued reference to FIGS. 6-8, the drive nut 116 includes an elongate body portion 117 and an engagement portion 118, connected therewith, which is configured for selective engagement with the piston. In the embodiment of FIGS. 1, and 5-8, the engagement portion 118 has an axial bore 119, axially aligned with and extending from the bore 114, with engagement projections or interior threads 120. The threads 120 selectively engage corresponding engagement projections or exterior helical threads 122, 124 on the piston 24, as best shown in FIGS. 9-10. It will be appreciated that the piston may alternatively be interiorly threaded to engage corresponding exterior threads on the drive nut.

As best shown in FIGS. 9 and 10, the threads 122, 124 on the piston 24 may take the form of at least one, more preferably two (or more) arcuately spaced helical flanges, which extend generally radially outward from opposite sides of the connection portion 28 of the piston 24. The flanges 122, 124 are configured for receipt into mating keyhole slots 126 in the connection portion 118 of the drive nut 116. FIG. 7 shows four keyhole slots 126, spaced approximately 90 degrees apart around the bore 119. The keyhole slots 126 provide access to the threads 120 on the drive nut. The piston flanges 122, 124 are thus received in a pair of opposed slots 126. To engage the drive nut 116 with the piston 24, the helical flanges 122, 124 are aligned with a pair of the slots 126 and the piston rotated about a quarter turn relative to the drive nut while holding the two parts firmly together. The flanges thus enter the bore 119 and engage the threads, thereby locking the drive nut to the piston against relative axial movement (i.e., inhibiting movement of the piston away from the drive nut in the dispensing direction or movement of the drive nut away from the piston in a direction opposite to the dispensing direction). As the drive nut 116 advances (i.e., in the dispensing direction) the piston 24 is pushed forwardly in the syringe cavity 18 to expel the medicament. In the event of an atmospheric depressurization, which tends to draw the piston 24 into the barrel 16 of the syringe, the engagement of the drive nut 116 with the connecting portion 118 resists this axial motion, inhibiting unintended administration of the medicament. When the drive nut is drawn in the opposite direction to the expelling direction by rotation of the lead screw 94 in an opposite direction to that used for advancement, the positive engagement of the drive nut with the piston causes the piston to be pulled outwardly of the syringe barrel 16.

An exterior surface 130 of the connecting portion 118 of the drive nut 116 is shaped to fit snugly in the syringe barrel chamber 18 to assist in maintaining axial alignment between the piston 24 and the drive nut during operation. FIG. 7 shows the exterior surface as defining a plurality of spaced flattened regions 132, which give the exterior surface a generally octagonal appearance, although other configurations are also contemplated. Additionally, the drive nut may include an axially extending alignment member 134 in the form of a tube, which extends forwardly from a shelf 135 of the connecting portion 118, as shown in FIG. 6. The alignment member 134 has an exterior cylindrical surface 136, which is shaped for snug receipt within the internal piston chamber 30 of the piston to assist in maintaining axial alignment (see FIG. 5). The walls of the chamber 30 and/or surface 136 may be tapered to ensure a snug receipt. This ensures accurate and smooth dispensing of the medicament from the barrel chamber 18. The alignment member 134 has an axial bore for receiving the lead screw 94 therethrough.

With reference to FIGS. 1 and 5, arcuately spaced projections 140 extend into the syringe barrel 16 adjacent the opening 22 (four projections in the illustrated embodiment). The projections 140 act as stops by engagement with an annular rim 142 on the piston (FIG. 9) to provide a user with an indication that the piston 24 is in its most extended position (illustrated in FIG. 1). This provides feedback to the user during filling of the syringe 14.

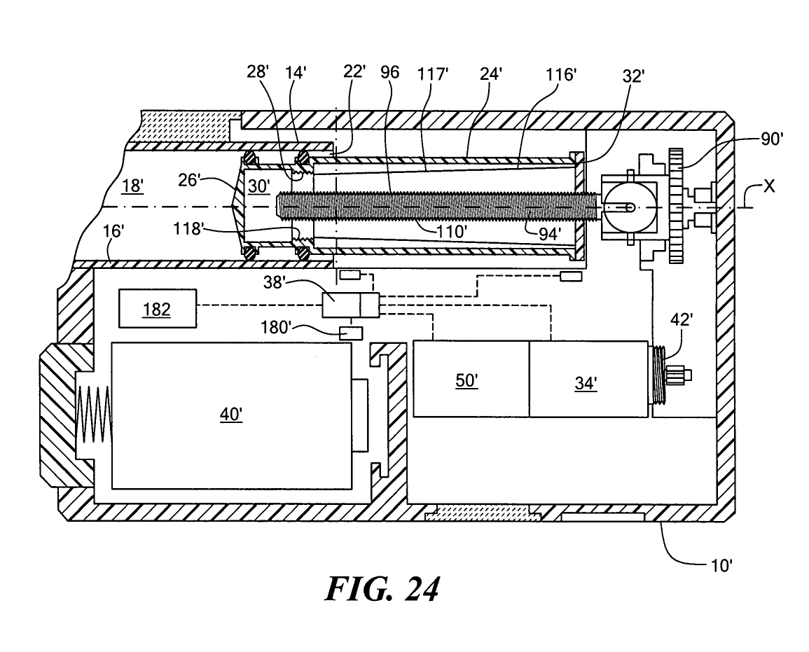

It will be readily appreciated that the exact shape of the drive nut 116 is not limited to that illustrated in FIGS. 1 and 6-8, but may be of any convenient shape to engage the piston. In an alternative embodiment, shown in FIG. 24, where similar components are numbered with a prime (′ ) suffix and new components are given new numbers, a drive nut 116′ includes a longitudinally extending conical body 134′, which is frustoconical in shape to be received within a correspondingly shaped interior chamber 30′ of the piston 24′ and thus provides guidance to the lead screw 94′ so that the piston 24′ moves longitudinally without excessive lateral wobbling. This ensures accurate and smooth dispensing of the medicament from the barrel chamber 18′.

In the embodiment of FIG. 24, the drive nut is threadably connected to the piston at 118′. Alternatively, the drive nut 116′ slides into and out of the piston 24′ without any form of positive engagement therewith (other than abutting contact). In this later embodiment, the drive nut 116′ is thus configured for one-way guiding of the piston 24′, i. e., the drive nut pushes the piston in a fluid expelling direction only. Unlike the embodiment of FIGS. 1 and 6-10, retraction of the drive nut 116′ (e.g., by rotation of the lead screw 94′ in an opposite direction to the driving direction) does not withdraw the piston 24′ from the barrel 16′.

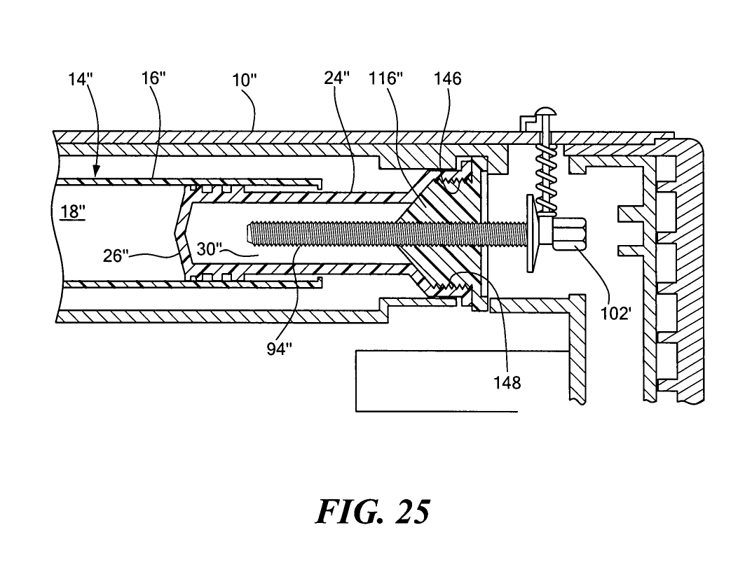

In yet another embodiment, shown in FIG. 25, where similar elements are numbered with a double prime (″) suffix, the drive nut 116″ is externally threaded at 146 to engage corresponding threads 148 on the internal piston chamber 30″. In this embodiment, the drive nut 116″ is configured for two-way driving of the piston 24″, as in the embodiment of FIG. 1. Retraction of the drive nut (e.g., by rotation of the drive shaft 94″ in an opposite direction to the driving direction) withdraws the piston 24″ from the barrel 16″.

In all the above-described embodiments, the lead screw is threaded and engages threads on the drive nut, such that, as the lead screw rotates, the drive nut advances.

With reference once more to the embodiment of FIGS. 6-8, the drive nut 116 includes a laterally extending flange 150 at a rearward end of the body portion 117, which defines a T-shape with opposed engagement surfaces 152. The engagement surfaces 152 of the flange 150 are guided by a guide element, which extends generally parallel with the drive nut 116. For example, the flange 150 is received through a longitudinal slot 154 in a guide element in the form of a hollow, tubular drive nut casing member 156 (FIG. 1). The casing member 156 slidingly accepts the drive nut 116 therein and may have an interior surface, which defines a plurality of guiding surfaces, such as flat planes or grooves for abutment with corresponding planes 158 and/or grooves 160 on the body portion 117. The slot 154, flange 150 and guiding surfaces 158, 160 cooperate to guide the body portion 117. In particular, the slot 154 of the guide element 156 contacts the engagement surface 152 (two engagement surfaces in the embodiment of FIG. 8) of the flange 150 and inhibits rotation of the flange 150 and the rest of the drive nut 116. In the embodiment of FIGS. 1 and 5, the guide element 156 defines an interior bore 158 having a generally rectangular cross section, which snugly receives the corresponding generally rectangular cross sectioned body portion 117. As the drive nut 116 is advanced, the piston 24 is driven into the barrel 16 of the syringe 14 and the medicament is expelled. Seals 164, such as o-rings, seal the gap between the piston 24 and the barrel 16 (FIG. 1). The guide element 156, 156′ is mounted to the housing 10 or to another rigid support within the housing, such as the gear box 54 (see FIG. 5).

In an alternative embodiment (not shown), the guide element 156 is in the form of a plate which extends parallel to the direction of travel of the drive nut.

As shown in FIG. 1, the travel of the drive nut 116 or piston 24 is preferably sensed by sensors 170, 172, which will be referred to herein as position sensors. For example, a first position sensor 170 detects when the drive nut 116 or piston 24 is in the “home” position (adjacent the driven end of the lead screw, as shown in FIG. 1). The sensor 170 may be an optical sensor, such as a visible light or infra-red sensor, mounted adjacent the home position of the flange 150 (or other suitable portion of the drive nut 116 or piston 24). The sensor 170 includes a transmitter (not shown), such as visible light or an infra-red transmitter, and a receiver (not shown) such as visible light or an infra-red receiver. When the flange 150 is adjacent the sensor 170, for example, within about one millimeter of the sensor, the infra-red radiation from the transmitter strikes a reflective portion 176 of the flange 150, such as a piece of reflective metal, and is returned to the receiver. Preferably, the casing 156 is light and or IR transparent, or has a suitably positioned aperture therein through which the light may travel. The sensor 170 detects when the signal is received and transmits a signal to the microprocessor controller 38 to indicate that the drive nut 116 is in the “home” position. In an alternative embodiment, the head 26 or other part of the piston 24 includes the reflective portion.

A second position sensor 172, analogous to the first sensor 170, is positioned close to, or adjacent to the “end” or “barrel empty” position of the reflective portion 176. The “end” position is the position that the reflective portion 176 is in when the piston head engages a dispensing end 178 of the barrel, i.e., where the flange 150 ends up when the piston 24 is depressed to the full extent of its travel. Preferably, the sensor 172's position is just before the end position (i.e., slightly to the left of the end position, in the arrangement of FIG. 1). The second sensor 172 signals the microprocessor-controller 38 when the reflective portion 176 is adjacent to the sensor 172, and the microprocessor portion of the microprocessor controller thereby recognizes that the drive nut 116 and piston 24 are approaching the end position. The controller portion of the microprocessor-controller instructs the motor 34 to cease advancing the shaft 42 and the piston 24 comes to a stop. In this way, the advancement of the piston 24 can be arrested before it hits a dispensing end 178 of the barrel 16, thereby avoiding potential damage to the drive system 36 or to the motor. This allows a “software” stop for the piston 24, rather than a “hard” stop that would result from physical contact between the components.

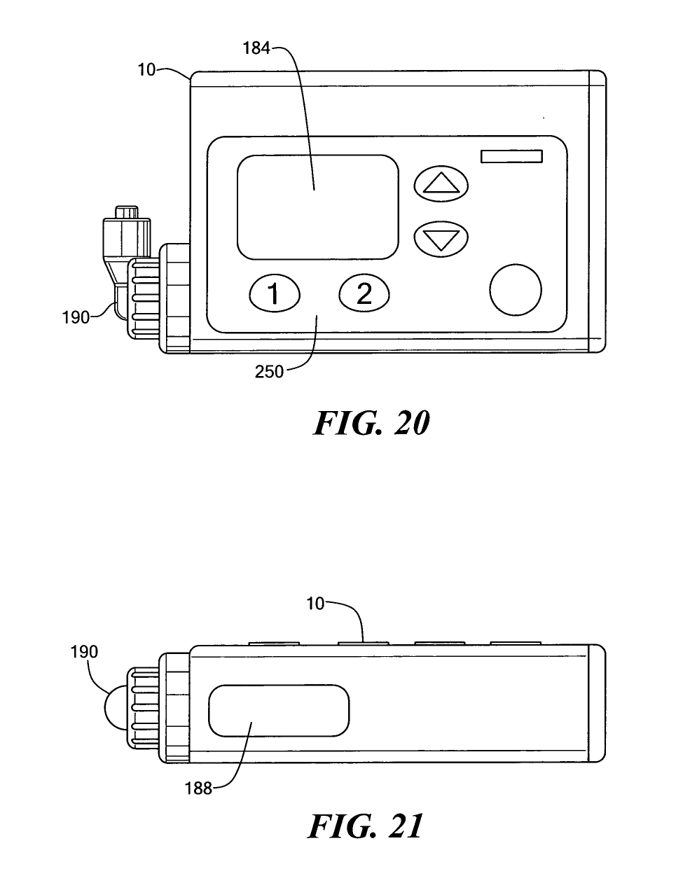

Alternatively, or additionally, the microprocessor may determine the position of the piston 24 from the signals received from the encoder 50 and by a calculation therefrom of the number of revolutions of the shaft 42. The microprocessor may use this determination as a check on the signals received from the second sensor 172, or to override the signal received from the second sensor when the two sets of signals are in conflict over the position of the piston 24. The microprocessor-controller 38 may signal an alarm, such as an audible alarm 180, a vibration alarm 182, and/or may send a message to an LCD or other visual display 184 (see FIG. 20) to indicate to the user or care provider that the syringe 14 is empty and needs to be refilled or replaced. The housing 10 may also include a window 188 (FIG. 21) for providing a visual indication to the user of the quantity of medicament still present.

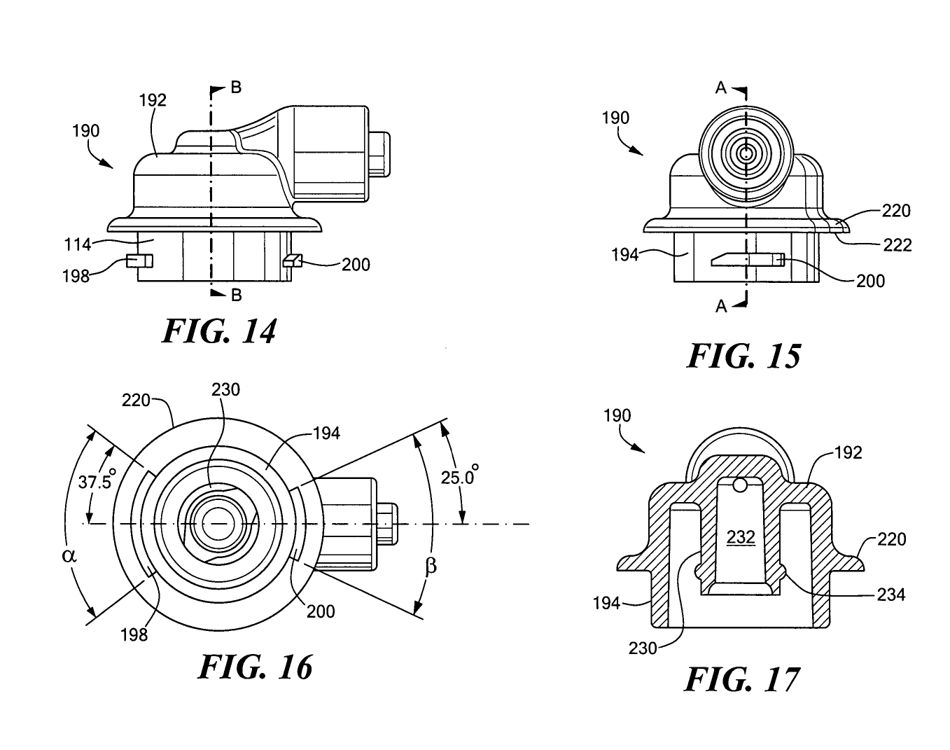

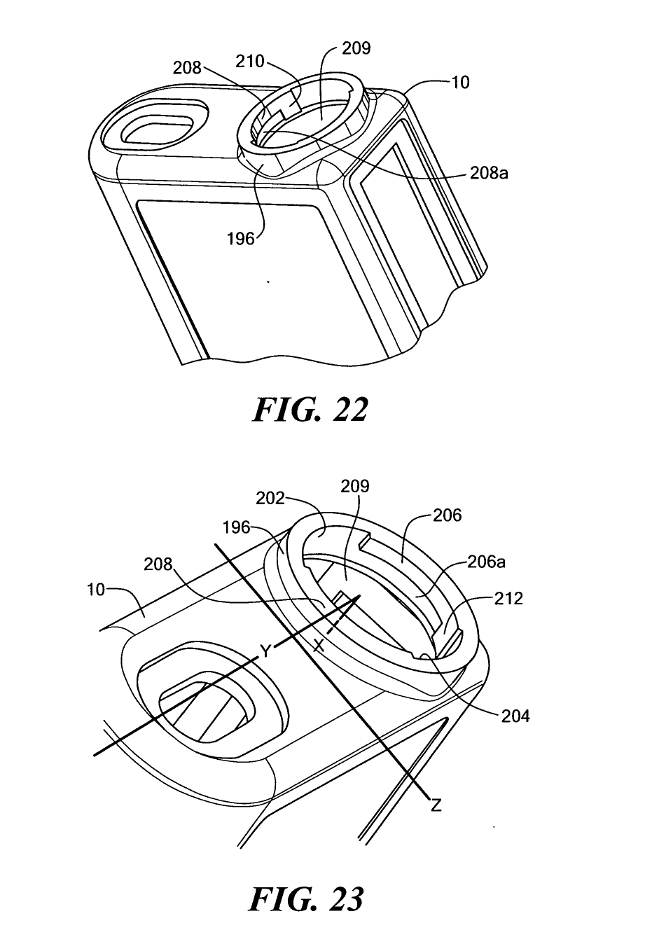

With reference once more to FIG. 1, and reference also to FIGS. 14-19, an external cap 190 secures the syringe 14 to the housing 10 and inhibits rotation of the syringe relative to the housing. The cap provides an aseptic fluid passageway between the syringe outlet 20 and an infusion line or other fluid line 191 (FIG. 19). In a preferred embodiment, best shown in FIG. 17, the cap 190 includes a top 192. A first annular skirt 194 extends from a periphery of the top and is exteriorly threaded or otherwise configured to engage an annular engagement portion 196, which protrudes forwardly of the housing 10, best shown in FIGS. 22 and 23. In particular, the skirt includes two circumferentially spaced projections in the form of tabs 198, 200, approximately 180 degrees apart, which extend radially outward from the skirt (i.e., generally perpendicular to the axis x of the syringe). Each tab 198, 200 thus defines a segment of an imaginary annulus around the skirt.

As best shown in FIG. 16, the tabs 198, 200 are of different lengths, such that their ends subtend different angles. For example, tab 198 is longer than tab 200, and subtends an angle a, which is greater than angle β subtended by the tab 200. For example, angle a may be from about 65°-90°, while angle β may be from about 30° to about 60°. The housing annular engagement portion 196 includes a pair of corresponding keyhole slots 202, 204 (FIGS. 22 and 23), which are similarly spaced and shaped for receipt of the two tabs 198, 200. The larger of the two tabs 198 fits only in the largest slot 202. The tabs 198, 200 thus provide a key for one directional receipt of the cap on the housing 10. To connect the cap to the housing, the tabs 198, 200 are aligned with the respective slots 202, 204 and pressed into the slots. The cap 190 is then rotated about a quarter turn, relative to the housing 10, to seat the tabs under adjacent annular rim segments 206, 208 of the engagement portion 196 in corresponding channels 206a and 208a. The rim segments 206, 208 and slots are arranged around a circular opening 209 in the housing, which is wide enough to receive the syringe 14 therethrough. Once the cap is twisted into the engaged position, with the tabs 198, 200 in the respective channels 206a, 208a, the cap is prevented or at least inhibited from being removed by puffing it outward, away from the housing 10.

As illustrated in FIGS. 22 and 23, the two channels are arranged in the same plane, perpendicular to the axis x of the housing. Thus, the cap syringe moves inward into the housing only during initial location of the tabs in the channels. Further rotation does not move the syringe further into the housing.

Preferably, a first stop 210 in the form of a projection extends radially inward of an interior wall of the engagement portion 196. FIG. 22 shows stop 210 as projecting radially inwardly at the end of the rim segment 208, thereby blocking the end of channel 208a. The stop 210 prevents rotation of the cap 190 in one rotational direction (anticlockwise in the illustrated embodiment), ensuring that the quarter turn rotation occurs in an opposite rotational direction (clockwise in the illustrated embodiment). A second stop 212 arrests the cap after the approximately quarter turn motion has been completed by engagement with the leading projection. It will be appreciated that both these functions could alternatively be provided by a single stop. The constrained one way quarter turn rotation provides a means for maintaining engagement of the piston 24 with drive nut 116, as will be described in greater detail below.

It will be appreciated that while the invention has been described with reference to the tabs 198, 200 as being on the cap 190, it is also contemplated that the tabs may be formed on the housing engagement portion 196 and the corresponding slots formed on the cap 190, rather than on the housing.

The annular skirt 194 includes a radial flange or shelf 220. A gasket 222 (FIG. 1), or other sealing member encircles the skirt 194. The radial shelf 220 holds the gasket 222 in sealing engagement with a portion 224 of the housing 10, which surrounds the engagement portion 196. The gasket inhibits the migration of contaminants into the housing 10. The cap 190 defines a second annular skirt in the form of a luer fitting 230 (FIGS. 1, 16-19), which depends from the top 192 and is spaced radially inward of the first skirt 194. The outlet port 20 of the syringe 14 fits snugly within a tapered interior passage 232 defined by the second annular skirt 230. Specifically, as shown in FIGS. 11-13, the syringe outlet 20 serves as a luer fitting for leak tight interconnection with fitting 230, and is configured for frictional fit in the tapered interior passage 232. The second skirt 230 is exteriorly threaded at 234 (FIG. 17) and threadably engages a corresponding annular interiorly threaded portion 236 of the syringe 14, which extends from the dispensing end 176 of the syringe 14, concentric with the outlet port 20, and is radially spaced therefrom.

A second luer fitting 240 (FIG. 19) optionally selectively connects the interior passage 232 of the cap with the infusion line 191. The second luer fitting 240 defines a second interior passage 244, which extends at right angles from the first interior passage 232. An annular, interiorly threaded portion 246 engages corresponding threads on the line 191. The quarter turn rotation of the cap which locks the cap to the housing ensures that the second luer fitting 240 is positioned as illustrated in FIGS. 20 and 21, i.e., lying generally parallel to axis y of the housing (FIG. 23) (which is perpendicular to axis x), such that no part of the luer fitting 240 extends outwardly beyond the housing in the direction of axis z (which is perpendicular to axes x and y). This reduces the chance that the luer fitting will become snagged by clothing, or the like, and thereby rotated to a disengaged position in which the cap can be inadvertently disconnected from the housing.

In another embodiment, a fixed or other form of connection may be made between the cap and the infusion line 191, whereby the infusion line is fluidly connected with the passage 230 and syringe outlet.

After a syringe 14 is filled with a medical solution, such as insulin, the syringe is screwed on to the first luer fitting 230 of the syringe cap 190. Alternatively, the user may use pre-filled, single use ampules. The piston 24 is optionally depressed to purge air bubbles from the cap and infusion line. The syringe 14 is then inserted into the housing 10 through the opening 226 and pushed inwardly, towards the drive nut, until the flanges 122, 124 are in contact with the drivenut. The cap 190, with the infusion line attached, is rotated clockwise, about a quarter turn, to engage the drive nut 116 with the piston 24, by rotating the piston relative to the drive nut so that the piston flanges 122, 124 enter the slots 126 on the drive nut and engage the threads 120. At this time, the tabs 198, 200 are outwardly spaced from their respective keyhole slots 206, 208. Once the piston flanges are engaged with threads 120, the cap tabs 198, 200 are inserted into their slots. This action causes the piston to be pushed into the syringe barrel slightly, clearing the line of air bubbles. The cap is then rotated by about a quarter turn in the same direction as that used for engagement of the flanges (clockwise in the illustrated embodiment) to lock the cap to the housing 10. In this rotational movement, the piston flanges 122, 124 rotate freely, relative to the drive nut, in the drivenut threads.

The hollow piston connection portion 28 slides over the sides of the cylindrical alignment member 134 of the drive nut (which is already retracted to its home position), and the piston is thereby guided into its correct position in the housing. When the syringe is fully inserted, the user programs the microprocessor-controller by way of a user-microprocessor interface 250, such as a keypad, touch screen, or other suitable interface (see FIG. 20). The user may select, for example, from a range of preprogrammed injection schemes or enter information, such as blood glucose levels, expected or actual carbohydrate intake, etc. in order for the microprocessor to calculate an appropriate infusion regimen. Or, the user may enter the amount of insulin to be infused in a selected time period. The infusion line may be connected with an infusion set (not shown) or other suitable infusion device for supplying the medication to the user's body.

The motor 34 rotates the drive shaft and the lead screw rotates, as described above. The interior threads on the drive nut 116 cause the lead screw and drive nut to begin to separate, pushing the drive nut and piston 24 in the dispensing direction.

Prior to making a connection between the infusion line 191 and an infusion set (not shown), the user preferably instructs the pump microprocessor-controller 38 to conduct a purge phase to clear the infusion line of air by passing a quantity of the medicament through the line. The user visually observes when the line is filled with the medicament and instructs the microprocessor 38 to halt the purge phase. The microprocessor detects that the drive nut flange 150 is no longer adjacent the first sensor 170 and also determines the quantity of medicament expelled during the purge phase from the signals from the encoder 50.

The microprocessor-controller 38 then controls the operation of the pump through the selected cycle. Using the information from the encoder 50, the microprocessor monitors the amount of medicament dispensed and provides a visual display to the user on the LCD display 184. The LCD displays black and white colors. However, the LCD display may also display at two or more colors, other than black and white. This may be a numerical display of the amount of insulin and/or in the form of a bar which decreases in size or in number of elements (similar to the indicator of battery level on a cellular phone) or other visual indication of decreasing medicament supplies. The controller uses this encoder-derived value as a second check as to when the medicament supply is about to run out. When the second sensor detects that the drive nut flange 150 is in the “empty” position, it signals the microprocessor-controller, which in turn stops the advancement of the motor. By way of the LCD display 184, the microprocessor-controller instructs the user to remove the syringe 14. Once the user has removed the syringe 14, the user signals the microprocessor that the syringe has been removed by making a suitable entry on the interface 250. The controller then reverses the direction of advancement of the motor 34 and the motor backs the drive nut 116 up to the “home” position. When the drive nut “home” position is detected by the sensor 170, the microprocessor instructs the user, by way of the LCD display 184, to insert a fresh syringe and the process is repeated.

In the event that an occlusion blocks the infusion line and reduces the flow of medicament to the user, an occlusion sensor system may be included. The detection of the occlusion can be accomplished by either software or hardware. Preferably, software determines the presence of an occlusion by processing signals received from encoder 50 (discussed in greater detail below). The occlusion sensor system detects the occurrence of an occlusion and signals an alarm to indicate to the user that the medicament is not being administered at the appropriate rate. In an alternate embodiment, an occlusion sensor is provided in hardware and may be included anywhere within the housing. For example, in one embodiment, as shown in FIG. 1, an occlusion sensor 260 is integral with the microprocessor-controller 38, although a separate occlusion sensor in an alternate location is also contemplated. The alarm can be the visual alarm, such as on the LCD display 184, the audible alarm 180, and/or the vibration alarm 182. The vibration alarm 182 preferably takes the form of a vibrating motor, which is connected with the microprocessor. The user may select which of the alarm functions is to be in operation, for example, by switching off the audible alarm 180 and activating the vibration alarm 182.

In one preferred embodiment, the occlusion sensor system operates by detecting stalling of the motor 34. If an occlusion in the line occurs, the pressure build up in the line inhibits advancement of the piston which, in turn, reduces or prevents rotation of the lead screw, gears and motor shaft, and causes the motor to stop or reduce its advancement. For example, the microprocessor-controller 38 detects if the signals from the encoder 50 indicate that the motor is not advancing or is advancing too slowly. In this embodiment of the occlusion sensor system, the microprocessor-controller counts how many signals are received from the encoder in a preselected time period and determines whether the number of signals is less than expected. Or, the microprocessor-controller detects an absence of any encoder signals in a preselected time period.

In an alternative embodiment of an occlusion sensor 260, shown in FIG. 4, a pressure transducer 270 or micro switch may be attached to a shaft portion 272 of the universal joint 90 to detect build-up of pressure in the lead screw 94 caused by the piston 24 being unable to traverse. The transducer signals the microprocessor-controller 38, which, if the pressure is above a preselected minimum pressure, signals the alarm, as with the other embodiment.

A digital clock or similar timing mechanism 280 is associated with the microprocessor controller 38. The user can instruct the microprocessor, by way of the keypad, to sound an alarm at one or more times. This provides a reminder to the user to take certain actions. For example, the user may input the times (e.g., four set times per day) at which he plans to take the medicament. At the specified times, the microprocessor generates an alarm, such as an audible, visual, or vibrational alarm, by activating one or more of audible alarm 180 or vibrational alarm 182. Alternatively, or additionally, the LCD display 184 displays a message, such as “take medication.” Other reminders, such as several times when blood sugar levels (or other body chemical) are to be tested, or a conventional alarm, for when the user should wake up, may also be programmed into the microprocessor-controller via the keypad. Preferably, the microprocessor-controller accepts at least a full day's schedule of reminders, e.g., four to six medication time reminders, four to six blood sugar test reminders, and one wake-up reminder.

The system also facilitates adjustable times of delivery. For some patients, it is desirable to provide a longer infusion time. The user can program the microprocessor controller, via the key pad to set the time of the delivery from a very short delivery time (depending on the amount to be infused), at which the motor operates at full speed, to a long delivery time, of, for example, twenty or thirty minutes, where the motor operates at a slower speed.

As can be seen, the arrangement of the motor 34 and drive shaft 42 in parallel with and adjacent to the syringe 14 and lead screw 94 makes good use of the space within the housing 10 and minimizes the overall length of the housing. Additionally, since neither the lead screw nor the drive shaft advances longitudinally in the housing 10 (both simply rotate), the housing 10 does not have to be enlarged to accommodate for longitudinal movement of these components. For example, a convenient size for the housing 10 is about 75 mm in length and about 45 mm in width.

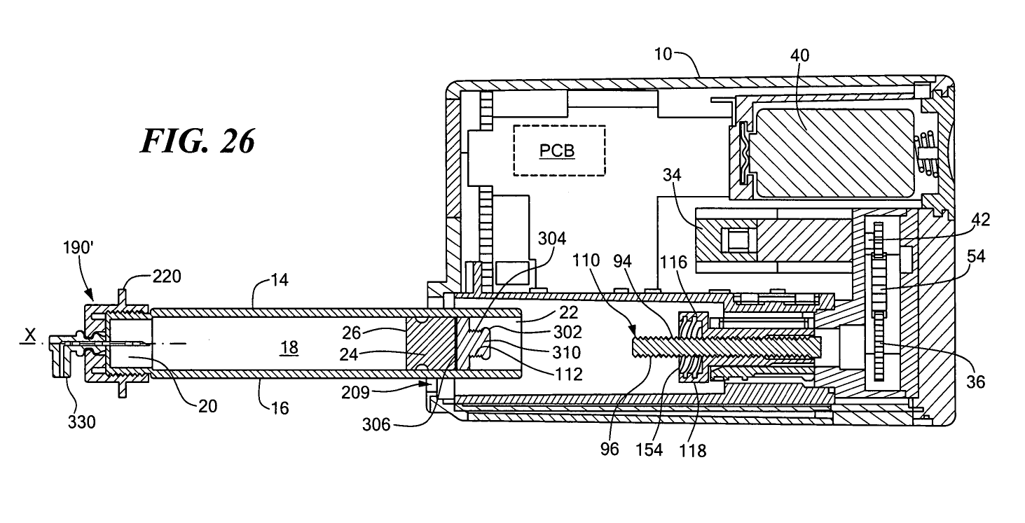

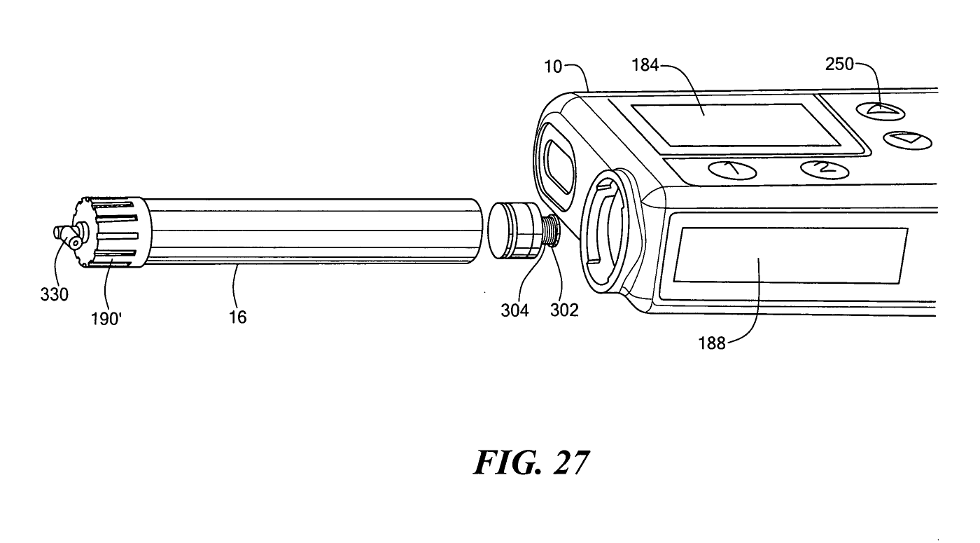

With reference to FIGS. 26 and 27, another embodiment of a portable pump system for use in an ambulatory injection system, such as an insulin injection system, is shown. The pump system is similar to that shown in FIGS. 1-23, except where otherwise noted. Similar elements are given similar numerals. The system includes a housing 10, which is designed to fit conveniently in the pocket of a user or to be attached to a belt clip. A cassette 14, such as a disposable or reusable syringe, is selectively received within the housing 10. FIG. 26 shows the syringe 14 partially inserted into the housing 10. The syringe 14 holds a supply of a medicament, such as insulin, for injection into a diabetic patient, or other user in need of the medicament. The syringe 14 includes a barrel 16, which defines an internal chamber 18 for holding the medicament, a dispensing outlet 20 in fluid communication with the internal chamber and connected with one end of the barrel 16, and an opening 22 at an opposite end of the barrel 16. A plunger or piston 24 is received within the barrel 16 via the opening 22 for reciprocal motion within the barrel 16 for selectively ejecting the medicament from the barrel. The piston 24 includes a head portion or cap 26, which seals the opening 22, and a connection portion 28, extending from the head portion.

Mounted within the housing 10, are a motor 34 and a drive system 36 for incrementally advancing the piston 24 to eject aliquots of the medicament, for example, according to a preprogrammed injection schedule. The motor, drive system, and microprocessor controller can be as described for FIG. 1, i.e., the motor 34 is under the control of a microprocessor-controller, which is preferably housed within the housing 10. Power for the motor and other operative components of the pump system is supplied by a replaceable/rechargeable battery 40, or other source of power. The motor 34 is preferably a stepper motor, which rotates in finite, small increments or steps. The drive system 36 includes a drive shaft 42, which is coupled to the motor so that it rotates a small portion of a revolution with each step of the motor. For example, the motor 34 may advance twenty steps to turn the drive shaft 42 one complete revolution, although other ratios may be contemplated or used without departing from the scope and intent of the present invention. As shown in FIG. 26, the drive shaft 42 is aligned generally in parallel with the longitudinal axis x of the syringe barrel 16 and piston 24 and rotates about an axis parallel with the x axis. It is also contemplated that the drive shaft may be coaxial with the piston axis of travel. However, an offset arrangement is desirable because of the ability to design a compact drive system.

As for FIG. 1, an encoder is operatively associated with an armature of the motor 34 to detect when the steps are occurring. For example, a two-phase encoder alternatively registers a “zero” or a “one” output with each successive step. The microprocessor-controller is equipped with processing software or hardware to detect the change in output of the encoder and thereby determine whether the motor 34 is advancing as instructed. The microprocessor-controller uses a measure of the number of motor steps to determine the rate and/or amount of medicament delivered. For example, it may instruct the motor to advance a selected number of steps over a certain time period, which equates to a determined volume of insulin ejected from the syringe in the selected time.

The drive shaft 42 drives a gearbox 54 comprising a series of gears similar to that shown in FIG. 2 to transfer driving movement from the motor to the piston. The number and size of the gears will depend on the desired ratio of drive shaft rotation to output rotation.

As for the embodiment shown in FIG. 4, the yoke element 90 is connected with a first portion, or driven end 92 of a threaded, rotatable shaft or lead screw 94. Thus, the rotations of the motor shaft 42 are transferred to the lead screw via the gear box 54 at a selected ratio, for example a ratio of from about 30:1-100:1 (30 to 100 rotations of the motor shaft for each rotation of the lead screw). A second, or distal end 96 (FIG. 26) of the lead screw 94 indirectly drives the piston 24 towards the chamber, so that the medicament is expelled.

In this embodiment, as for that of the embodiment of FIG. 1, the lead screw 94 is received longitudinally within a chamber of a drivenut or piston drive member 116 and extends generally parallel to the drive shaft 42. As shown in FIG. 4, the driven end 92 may comprise a ball and pin member 98, which is received in a slotted opening 100 in the yoke element 90. Other engagement methods, which transfer the rotation of the yoke member to the lead screw, are also contemplated, such as a fitting comprising a hexagonal pin (not shown) on the driven end 92, which is received in a corresponding hexagonal socket (not shown) in the universal joint 90 (not shown). Alternatively, the yoke 90 and lead screw 94 may be formed as a single component. The lead screw can be fixed to the gear box or disconnectable.

With continued reference to FIGS. 26-27, the drive nut 116 includes an elongate body portion 117 and an engagement portion 118, connected therewith, which is configured for selective engagement with the piston. In the embodiment of FIGS. 26 and 27, the engagement portion 118 is interiorly threaded, similar to the drivenut of FIG. 1, to engage corresponding exterior threads or a flange 302 on a connector 304. It will be appreciated that the drivenut may alternatively be exteriorly threaded to engage corresponding interior threads on the drive nut.

The connector 304 is selectively attached to the piston 24. In one embodiment, the connector comprises an adhesive layer 306, such as a double sided adhesive tape, although alternative means of selectively engaging and disengaging the connector from the piston are contemplated, such as threaded engagement. Alternatively, a piston as shown in FIG. 1 is employed, obviating the need for a connector. The adhesive layer 306 may be initially attached either to the piston 26 or to the connector 304 and covered with a release layer, prior to use. When it is desired to attach the connector to the piston, the release layer is removed and the two components joined with the adhesive layer. Optionally, the adhesive is of the type which allows the two parts to be pulled apart, after the syringe is empty such that the connector and adhesive layer can be reused with another syringe.

The connector 304 is similarly configured, at its rearward end, to the piston of FIG. 1 in that it has an internally threaded cavity 310 for threadably receiving the end 96 of the lead screw 94. The adhesive 306 effectively locks the connector 304 to the piston such that the piston is locked against relative axial movement (i.e., inhibiting movement of the piston away from the connector in the dispensing direction or movement of the drive nut away from the piston in a direction opposite to the dispensing direction). As the drive nut 116 advances (i.e., in the dispensing direction) the piston 24 is pushed forwardly in the syringe cavity 18 to expel the medicament. In the event of an atmospheric depressurization, which tends to draw the piston 24 into the barrel 16 of the syringe, the adhesive engagement of the connector 304 with the piston resists this axial motion, inhibiting unintended administration of the medicament. When the drive nut is drawn in the opposite direction to the expelling direction by rotation of the lead screw 94 in an opposite direction to that used for advancement, the positive engagement of the drive nut with the piston via the connector 304 causes the piston to be pulled outwardly of the syringe barrel 16.

As for the embodiment of FIGS. 1 and 5, arcuately spaced projections similar to projections 140 may extend into the syringe barrel 16 adjacent the opening 22 (four projections in the illustrated embodiment to act as stops by engagement with an annular rim 142 on the piston similar to that shown in FIG. 9 to provide a user with an indication that the piston 24 is in its most extended position, as illustrated in FIG. 1. This provides feedback to the user during filling of the syringe 14.

With reference once more to the embodiment of FIGS. 26-28, the drive nut 116 may include a laterally extending flange similar to flange 150 for engagement with a guide element 156 similar to that shown in FIGS. 6-8.

As for the embodiment of FIG. 1, the travel of the drive nut 116 or piston 24 is preferably sensed by sensor similar to sensors 170, 172.

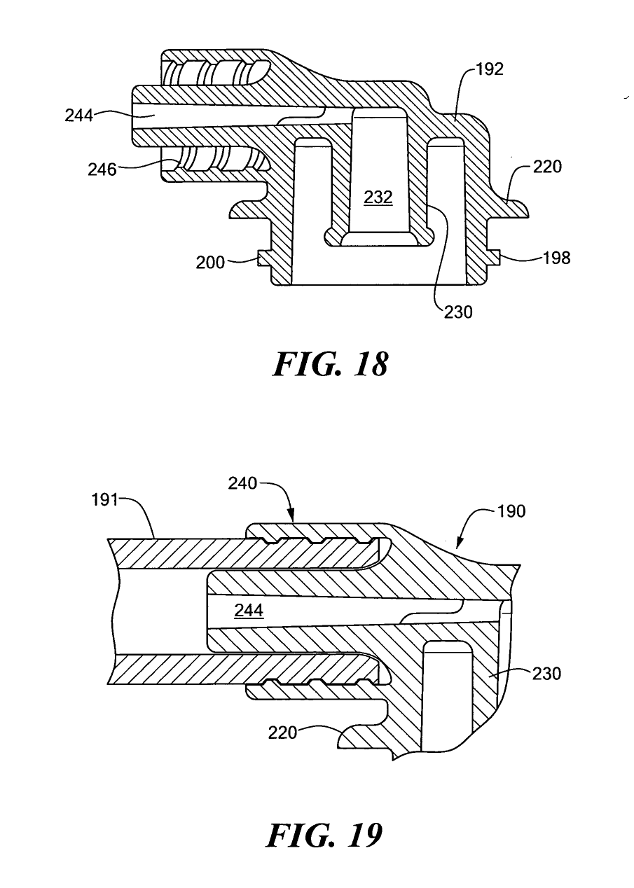

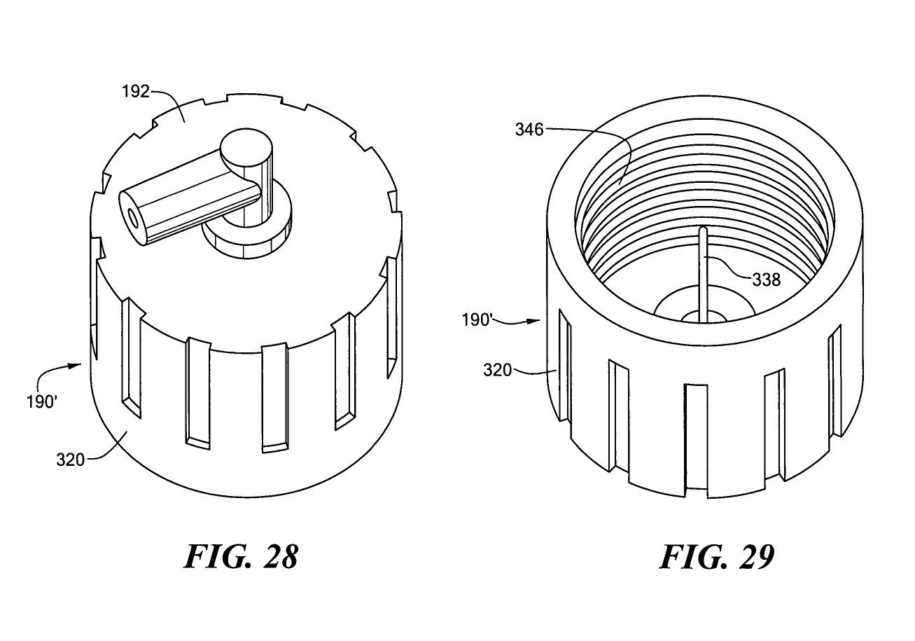

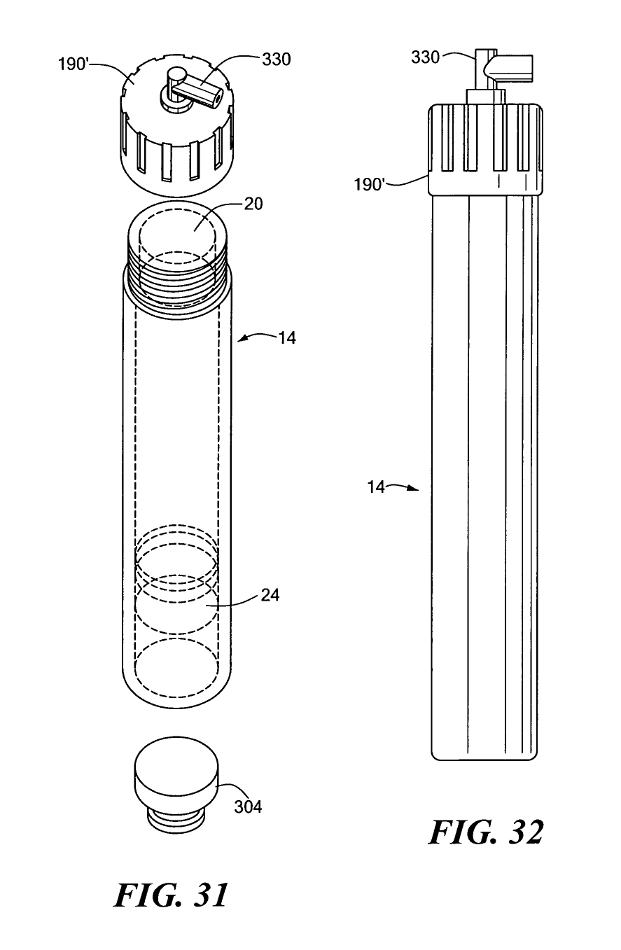

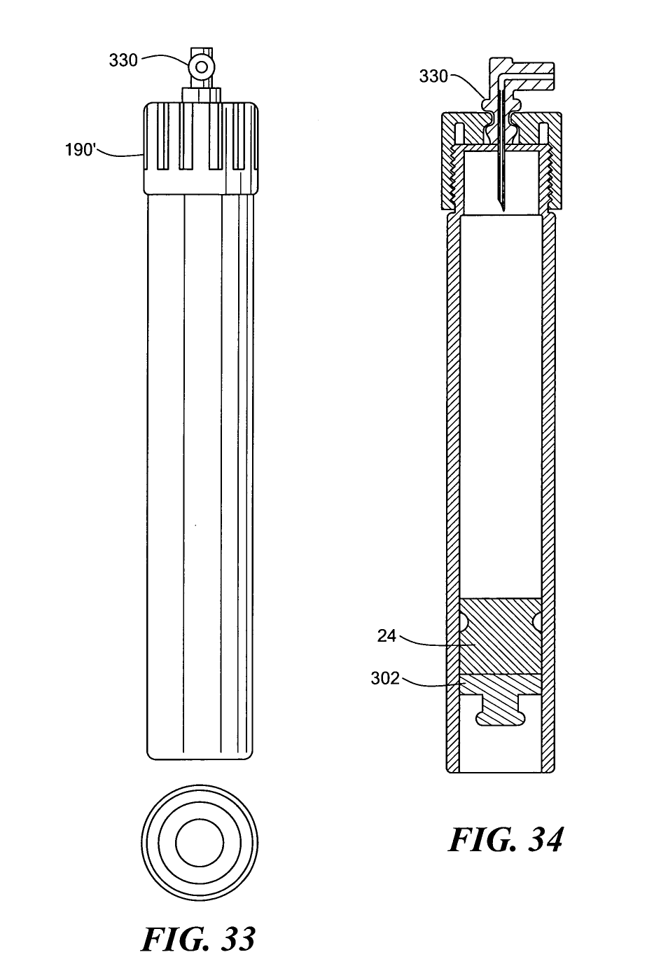

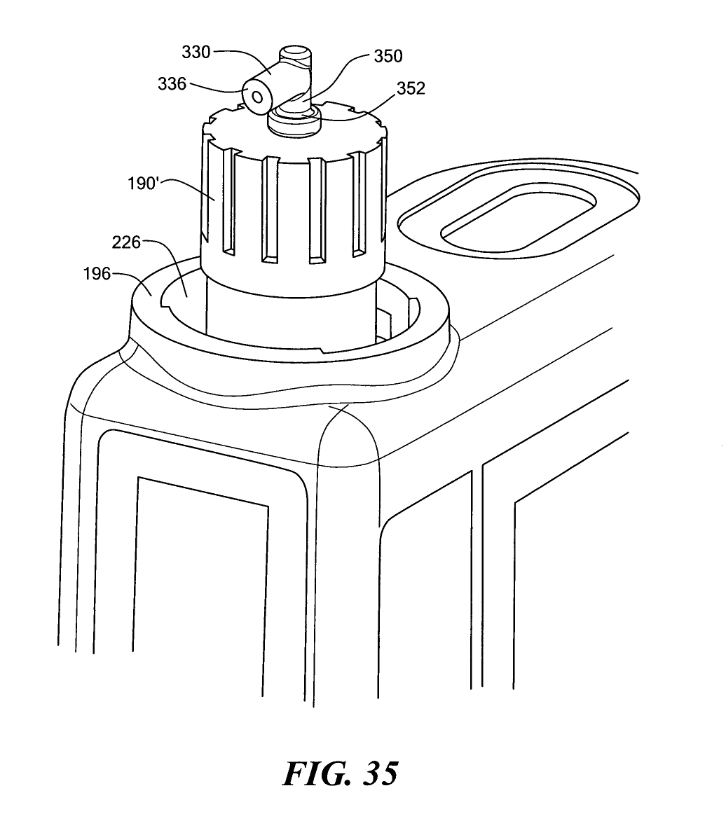

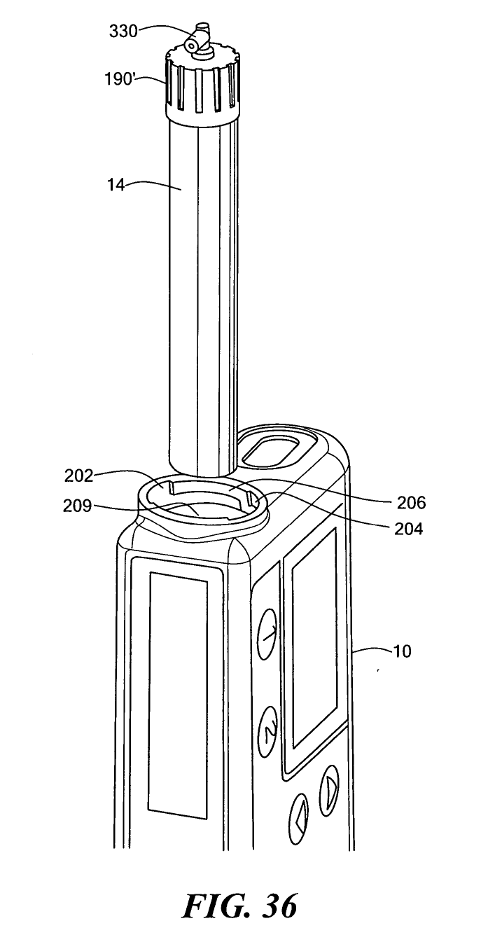

With reference once more to FIG. 26-27, and reference also to FIGS. 28-36, an external cap assembly comprising a cap 190′ is selectively attached to the syringe. The cap assembly provides an aseptic fluid passageway between the syringe outlet 20 and an infusion line or other fluid line similar to line 191 of FIG. 19. As shown in FIG. 28, the cap 190′ includes a top 192 with a skirt 320. The skirt 320 extends from a periphery of the top and is interiorly threaded. Optionally, the skirt 320 is exteriorly threaded or otherwise configured with tabs similar to tabs 198, 200 to engage an annular engagement portion similar to engagement portion 196, shown in FIGS. 18, 22 and 23.

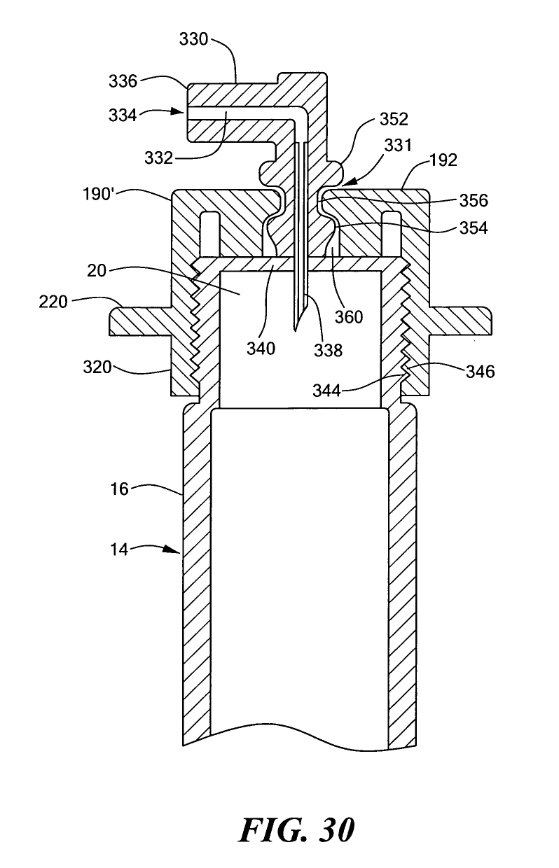

The annular skirt 320 may include a radial flange or shelf 220 as shown in FIG. 30 for engaging a gasket similar to gasket 222 of FIG. 1.

A rotatable hub 330 is axially mounted through a central aperture 331 in the top of the cap 190′ best shown in FIGS. 28 and 30. The rotatable hub defines a through passage between an outlet port 334, located at a connection end 336 of the rotatable hub 330, and a needle 338, which is configured for piercing a pierceable closure 340 on the end 20 of the syringe barrel 16. The outlet port 20 of the syringe 14 is exteriorly threaded at 344 to engage corresponding interior threads 346 on the cap skirt when the cap is threaded on to the syringe. During the threading procedure, the needle pierces the closure 340.

The connection portion 336 may be integrally connected with a line 191 which supplies insulin to a user. Alternatively, the connection end 336 may be configured, such as with a luer fitting similar to luer fitting 240 (FIG. 19) to connect on to the line 191

The connection portion 336 of the rotatable hub is rotatable, relative to the cap 190′, about axis x to avoid tube tangling. In particular, the rotatable hub includes a mounting portion 350, which extends perpendicular to the connection portion 336, generally axially along axis x. The mounting portion 350 defines a first projection 352 and a second projection 354, which are axially spaced by a groove 356. The groove is shaped to fit snuggly between corresponding projections which define the cap top opening 331. The rotatable hub can thus be installed in the cap 190′ by pushing the mounting portion 352 through the opening 331 until the projection 354 snaps past the opening and the groove 356 is seated in the opening. The projection 354 is thereby seated in a chamber 360, of slightly wider lateral dimension than the opening 331. The two projections 352 and 354 resist removal of the hub from the cap during normal use but allow rotation of the hub relative to the cap. The needle 338 fits tightly into the passage 332 in the mounting portion, to define a leak tight connection with the passage. In one embodiment, the needle 338 is integrally formed with the rest of the hub. In another embodiment, the needle is molded into the rest of the hub such that the two parts become one during molding.

In an alternative embodiment, the hub is fixed in position, and does not rotate, relative to the cap.

The syringe 14 may be pre-filled with insulin or other injectable liquid at the factory and sealed with the closure 340. Alternatively, a user fills the syringe from a bulk vial and then fits the closure 340 to the syringe.

After a syringe 14 is filled with a medical solution, such as insulin, the cap 190′ is screwed on to the syringe. The connection member is then adhesively attached to the exposed end of the piston 24. The piston 24 is optionally depressed to purge air bubbles from the cap and infusion line. The syringe 14 is then inserted into the housing 10 through the opening 226 and pushed inwardly, towards the drive nut, until the flange 302 snap fits or threadably connects with the portion 118 of the drivenut.

Once the flange 302 is engaged with portion 118, the cap may be engaged with the housing, in a similar manned to that illustrated in FIGS. 18 and 23,e.g., with tabs (not illustrated) similar to flanges or tabs 198, 200, which engage slots 202,204 around the housing opening 209. This action causes the piston to be pushed into the syringe barrel slightly, clearing the line of air bubbles. The cap is then rotated by about a quarter turn in the same direction as that used for engagement of the flanges (clockwise in the illustrated embodiment) to lock the cap to the housing 10.

The hollow portion 310 of the connector 304 receives the lead screw 94. When the syringe is fully inserted, the user programs the microprocessor-controller by way of a user-microprocessor interface 250, such as a keypad, touch screen, or other suitable interface (see FIG. 20). The user may select, for example, from a range of preprogrammed injection schemes or enter information, such as blood glucose levels, expected or actual carbohydrate intake, etc. in order for the microprocessor to calculate an appropriate infusion regimen. Or, the user may enter the amount of insulin to be infused in a selected time period. The infusion line may be connected with an infusion set (not shown) or other suitable infusion device for supplying the medication to the user's body.

The motor 34 rotates the drive shaft and the lead screw rotates, as described above. The interior threads on the drive nut 116 cause the lead screw and drive nut to begin to separate, pushing the drive nut and piston 24 in the dispensing direction.

Prior to making a connection between the infusion line 191 and an infusion set (not shown), the user preferably instructs the pump microprocessor-controller 38 to conduct a purge phase to clear the infusion line of air by passing a quantity of the medicament through the line. The user visually observes when the line is filled with the medicament and instructs the microprocessor 38 to halt the purge phase. The microprocessor detects that the drive nut flange 150 is no longer adjacent the first sensor 170 and also determines the quantity of medicament expelled during the purge phase from the signals from the encoder 50.

The microprocessor-controller 38 then controls the operation of the pump through the selected cycle. Using the information from the encoder 50, the microprocessor monitors the amount of medicament dispensed and provides a visual display to the user on the LCD display 184. Preferably, LCD display is a color LCD display, which displays at least three colors, other than black and white. This may be a numerical display of the amount of insulin and/or in the form of a bar which decreases in size or in number of elements (similar to the indicator of battery level on a cellular phone) or other visual indication of decreasing medicament supplies. The controller uses this encoder-derived value as a second check as to when the medicament supply is about to run out. When the second sensor detects that the drive nut flange 150 is in the “empty” position, it signals the microprocessor-controller, which in turn stops the advancement of the motor. By way of the LCD display 184, the microprocessor-controller instructs the user to remove the syringe 14. Once the user has removed the syringe 14, the user signals the microprocessor that the syringe has been removed by making a suitable entry on the interface 250. The controller then reverses the direction of advancement of the motor 34 and the motor backs the drive nut 116 up to the “home” position. When the drive nut “home” position is detected by the sensor 170, the microprocessor instructs the user, by way of the LCD display 184, to insert a fresh syringe and the process is repeated.

In the event that an occlusion blocks the infusion line and reduces the flow of medicament to the user, an occlusion sensor system 260 similar to that described for FIG. 1 or 4 detects the occlusion and signals an alarm to indicate to the user that the medicament is not being administered at the appropriate rate

A digital clock or similar timing mechanism similar to clock 280 is associated with the microprocessor controller 38, as shown in FIG. 1.

As will readily be appreciated, the infusion pump and drive system of the present have applications outside the medical field and are not limited to use in an infusion system.

The invention has been described with reference to the preferred embodiment. Obviously, modifications and alterations will occur to others upon reading and understanding the preceding detailed description. It is intended that the invention be construed as including all such modifications and alterations insofar as they come within the scope of the appended claims or the equivalents thereof.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A liquid delivery system comprising:
a housing which accommodates a syringe containing the liquid;
means for expelling a liquid from the syringe carried by the housing;
a cap which selectively connects the syringe with the housing and provides a fluid passage between the syringe and a fluid line when the fluid line is connected with the cap, the cap including means for selectively connecting the cap with the syringe;
at least two spaced projections on one of the cap and the housing; and
at least two spaced slots on the other of the cap and the housing which receive the projections, such that when the projections are positioned in the slots, the cap is moved relative to the housing in a locking direction to lock the cap to the housing.

2. The system of claim 1, wherein the projections extend radially outwardly from an annular skirt of the cap.

3. The system of claim 1, wherein a first of the projections is configured for receipt only in a first of the slots.

4. The system of claim 3, wherein the first projection subtends a larger angle than a second of the projections.

5. The system of claim 1, wherein when the projections are positioned in the slots, the cap is rotatable only in the locking direction.

6. The system of claim 1, further comprising a stop associated with the housing, wherein when the projections are positioned in the slots, the stop resists rotation of the cap in an unlocking direction.

7. The system of claim 6, further including a second stop associated with the housing which limits rotation of the cap relative to the housing to less than one revolution.

8. The system of claim 1, further including a second stop associated with the housing which limits rotation of the cap relative to the housing to about a quarter of a revolution.

9. The system of claim 1, wherein the means for expelling comprises:
a motor carried by the housing; and
a drive system, operatively connected with the motor, which advances a piston of the syringe to expel liquid from a barrel of the syringe, the drive system including:
a threaded rotatable shaft; and
a piston drive member, which linearly advances the piston, the drive member defining a threaded portion which engage threads of the shaft, the piston drive member advancing linearly as the shaft rotates.

10. The liquid delivery system of claim 9, wherein the drive member defines an engagement portion which selectively engages an engagement portion of the piston to lock the drive member to the piston against relative axial movement.

11. The liquid delivery system of claim 10, wherein the drive member engagement portion engages the piston engagement portion as the cap is rotated relative to the housing in a locking direction.

12. The liquid delivery system of claim 9, wherein the drive member engagement portion defines threads which threadably engage corresponding threads of the piston engagement portion.

13. The liquid delivery system of claim 9, wherein the drive member engagement portion defines opposed keyhole slots which each receive a helical thread of the piston engagement portion.

14. The liquid delivery system of claim 9, wherein the piston drive member includes a flange which is constrained by a guiding member associated by the housing which resists rotation of the drive nut such that it advances axially as the shaft rotates.

15. The liquid delivery system of claim 9, further comprising:
a first position sensor which detects when at least one of the piston and the piston drive member is in a first position; and
a second position sensor which detects when the at least one of the piston and the piston drive member is in a second position, linearly spaced from the first position.

16. The liquid delivery system of claim 15, wherein when the at least one of the piston and the piston drive member is in the first position, the piston is spaced from a liquid outlet of the syringe through which the liquid is dispensed and wherein when the at least one of the piston and the piston drive member is in the second position, the piston is closely adjacent the liquid outlet of the syringe.

17. The liquid delivery system of claim 9, wherein the motor is a stepper motor and further including:
an encoder which detects step movements of the motor; and
occlusion sensor means which detects when there is an occlusion in the delivery system, the occlusion sensor means receiving signals from the encoder and determining an occlusion from a reduction in a speed of the step movements.

18. The system of claim 1, wherein the means for selectively connecting the cap with the syringe includes a luer connection.

19. The system of claim 1, further including a connector for connecting the piston with the drivenut.

20. The system of claim 19, wherein one of the piston and the drivenut includes a layer of adhesive, such as double sided tape, for adhesively attaching the piston to the drivenut.

21. The system of claim 17 further comprising a power supply for powering the stepper motor, the power supply being programmable to allow for adjustment of the torque of the stepper motor.

22. A liquid delivery system comprising:
a housing which accommodates a syringe containing the liquid;
means for expelling a liquid from the syringe carried by the housing;
a cap assembly which selectively connects the syringe with a fluid line, the cap assembly including a cap for connecting the cap assembly with the syringe and a rotatable hub which provides a fluid passage between the syringe and the fluid line.

23. The system of claim 22, wherein the rotatable hub includes a needle, which defines a portion of the passage, for piercing a closure on the syringe when the cap assembly is connected to the syringe.

24. The system of claim 22, wherein the cap includes an opening which receives the rotatable hub in a snap fit connection.

25. The system of claim 22, further including:
means for selectively connecting the cap to the housing.

26. A cap assembly for connecting a syringe to an infusion line, the cap assembly comprising:
a threaded cap for selective interconnection with an outlet port of an associated syringe; and
a rotatable hub which rotates relative to an axis of the cap and is configured at a first end for connection with an infusion line or is integrally formed therewith, a second end of the rotatable hub being received through an opening in the cap and defining a needle for piercing a closure on the associated syringe.

27. A method of assembling an infusion system comprising:
coupling a cassette, containing a liquid to be infused, to a cap;
mounting the cap on a housing such that the cassette is received within the housing, the mounting step including:
engaging first and second projections on one of the cap and the housing with first and second slots on the other of the cap and the housing, the projections being configured such that the first projection is received only in the first slot;
rotating the cap relative to the housing in a locking direction to lock the cap to the housing.

28. The method of claim 27, wherein the step of rotating includes rotating the cap relative to the housing by less than a complete revolution until a stop inhibits further rotation of the cap relative to the housing.

29. The method of claim 27, wherein the step of rotating the cap relative to the housing in a locking direction engages a piston of the cassette with a drive member such that the piston is locked against axial movement relative to the drive member.

30. The method of claim 27, wherein the cap includes a rotatable hub which defines a passage terminating at one end with a needle and the method includes:
piercing a closure on the cassette with the needle.
